(19) United States
(12) Patent Application Publication
(10) Pub. No.: US 2002/0141964 A1
(43) Pub. Date: Oct. 3, 2002

(54) Composition for arresting the flow of blood and method

(76) Inventors: James A. Patterson, Sarasota, FL (US); John A. Thompson, Nassau, FL (US); Talmadge Kelly Keene, Apollo Beach, FL (US)

Correspondence Address: Charles J. Prescott, Charles J. Prescott, P.A., Suite 115, 2033 Wood Street, Sarasota, FL, 34237-7926, US

(21) Appl. No.: 09/766,513

(22) Filed: Jan. 19, 2001

(51) Int. Cl. A61K 31/74; A61K 33/14; A61K 33/32; A61K 33/26

(52) U.S. Cl. 424/78.1; 424/661; 424/648; 424/641

(57) ABSTRACT

A hemostatic agent for arresting the flow of blood and other protein containing body fluids flowing from an open wound and for promoting wound healing. One embodiment is directed to a substantially anhydrous admixture of an oxyacid salt and a hydrophilic proton donor which will hydrate in the presence of blood and body fluid to produce cations to promote blood clotting. The preferred oxyacid salts are alkali and alkaline earth salts of transition metals and halogen oxyacids with oxidizing capabilities sufficient to accelerate blood clotting. Another embodiment of the invention includes the compound containing an oxysalt plus a hydrophilic polymer such as carboxy methylcellulose, polyvinyl, alcohol, an alginate, and all soluble gums. Still another embodiment of the invention includes the compound formed of an oxyacid salt in combination with a hydrophilic proton donor and a solid desiccant which further accelerates blood coagulation reaction rates. The cation exchange material or an admixture of an alkali metal oxyacid salt plus acidic inorganic salt produces a scab or protective coating over the wound for protection and enhanced healing. Oxygen produced during the reaction substantially reduces the level of bacteria, virus and fungus at the wound.

### BACKGROUND OF THE INVENTION

1. Scope of Invention

This invention relates generally to topically applied agents for promoting blood clotting to arrest blood flow from an open wound, and more particularly to a method of applying an anhydrous composition which may be mixed just prior to its application directly over an open bleeding wound or a wound from which body fluid is flowing to accelerate flowing blood and body fluid clotting and enhance healing.

2. Prior Art

In addition to conventional bandages, adhesive means, compresses and the like which are applied with pressure directly against a bleeding open wound, considerable effort has been directed toward the development of chemical agents in various forms which accelerate or enhance the coagulation of blood flowing from an open wound to arrest blood flow. Many of these agents are in the “clotting chain”, i.e., fibrinogen, thrombin, Factor VIII and the like. Others are based upon the use of collagens. Potassium permanganate alone is also known to be a weak astringent, but causes intolerable severe stinging pain at the wound.

Edwardson, in U.S. Pat. Nos. 5,763,411, 5,804,428, and 5,962,026, for example, teaches the use of fibrin in conjunction with a solid support in the '411 patent, and as an enzyme free sealant in the '428 patent, and as a solid composition substantially free of catalytic enzymes.

Three U.S. Patents invented by Martin, U.S. Pat. Nos. 5,692,302, 5,874,479 and 5,981,606, are generally directed to the use of pyruvate in combination with fatty acids and an oxidant as a therapeutic wound healing composition.

Stilwell, in U.S. Pat. No. 5,484,913 teaches the use of calcium-modified oxidized cellulose to promote faster hemostasis. In U.S. Pat. No. 5,474,782, Winter, et al. teaches a wound healing composition or its salt present in a pharmaceutically acceptable carrier, the preferred embodiment being a salt of sodium. Winter provides a wound dressing with a taspine compound for promoting healing rather than clotting.

In U.S. Pat. No. 2,163,588, Cornish teaches a wound pad having very fine fibers carrying a viscous agent and a septic for arresting and clotting blood flow. Eberl, et al., in U.S. Pat. No. 2,688,586, teaches an improved hemostatic surgical dressing with alginic acid as a clotting agent. Masci, et al. in U.S. Pat. Nos. 2,772,999 and 2,773,000 also teaches hemostatic surgical dressing including a pad and free acid cellulose glycolic acid.

A patent for another hemostatic wound dressing is taught by Shelley in U.S. Pat. No. 3,206,361 having an active agent in the form of methylaminoacetocatechol hydrochloride. Likewise, Anderson, in U.S. Pat. No. 3,328,259, another wound dressing containing a film of cellulose glycolic acid ether is provided as the hemostatic agent.

The hemostatic agent taught by Sugitachi, et al. as disclosed in U.S. Pat. No. 4,265,233 is blood coagulation Factor VIII plus either fibrin or thrombin. A ready-to-use bandage is taught by Altshuler in U.S. Pat. No. 4,363,319 which also contains thrombin as an active agent, the bandage all of which is contained within a sealed package.

Invented by Lindner, et al., a wound pad which is impregnated with tissue-compatible protein such as collagen and lyophilized Factor XIII, thrombin and fibrinogen, are taught in U.S. Pat. No. 4,600,574. The use of collagen as a hemostatic agent within a pad that has been freeze-dried is taught by Sawyer in U.S. Pat. No. 4,606,910.

In U.S. Pat. No. 4,616,644, Saferstein, et al. teaches the use of an adhesive bandage with high molecular weight polyethylene oxide applied to the surface of the perforated plastic film wound release cover of the bandage to arrest blood flow from minor cuts. Yet another hemostatic agent including a carrier in the shape of a flake or fiber having thrombin and Factor XIII affixed thereto is taught by Sakamoto in U.S. Pat. No. 4,655,211. The use of an ultra-pure, clean thrombin solution as a hemostatic agent is taught in U.S. Pat. No. 5,525,498 invented by Boctor. Two recent patents invented by Pruss, et al., U.S. Pat. Nos. 5,643,596 and 5,645,849 both teach the use of hemostatic dressings which incorporate thrombin and epsilon aminocaproic acid (EACA) and calcium chloride on gelatin.

An absorbable spun cotton-like topical hemostat is taught by Shimuzu, et al. in U.S. Pat. No. 5,679,372. This disclosure is directed to an absorbable dressing made of acetocollagen fibers which are innately adhesive to a bleeding surface. In a patent to Bell, et al, U.S. Pat. No. 5,800,372, a dressing made of microfibrillar collagen and a superabsorbant polymer provides both blood absorption and clotting inducement.

A previous U.S. patent ______ co-invented by James A. Paterson and J. A. Thompson, also co-inventors of the present case, teaches utilizing an improved ion exchange resin, preferably in the form of a styrene divinylbenzene copolymer which has been sulfonated. The collective teaching of making this prior art resin is to be found in an earlier patent to co-inventor, Patterson, U.S. Pat. No. 4,291,980. This manufacturing method disclosed in the '980 patent was based at least in part on the production of spherical beads comprised of copolymer styrene and divinylbenzene as taught in U.S. Pat. Nos. 2,366,007 and 3,463,320. An improvement better adapting this resin to the present invention is in the form of substantially reduced cross-linking down to about 0.25%.

Another primary aspect of the above-referenced previous invention incorporated a salt ferrate, preferably potassium ferrate (2K₂FeO₄). The teaching of a process for producing alkaline metal ferrates is taught by another co-inventor, Thompson, in U.S. Pat. No. 4,545,974. This teaching is also incorporated herein by reference.

It is submitted that the above-referenced prior art, either taken individually or collectively in any combination thereof fail to teach a flowing blood or body fluid clotting agent which includes an admixture of an oxyacid salt, in combination with a cation exchange resin, an organic acid or an acidic inorganic salt, which reacts with the blood or protein in blood to accelerate coagulation and clotting of the blood. Moreover, the utilization of an insoluble cation exchange material, in recombination with the oxyacid salt, additionally produces a protective covering over the wound and also produces oxygen which acts as an antibacterial, antiviral and antifungal agent. Further, the presence of a selected hydrophilic proton donor neutralizes hydroxide radicals as clotting occurs so as to eliminate any substantial stinging sensation.

### BRIEF SUMMARY OF THE INVENTION

This invention is directed to a hemostatic agent for arresting the flow of blood and other protein containing body fluids flowing from an open wound and for promoting wound healing. One embodiment is directed to a substantially anhydrous admixture of an oxyacid salt and a hydrophilic proton donor which will hydrate in the presence of blood and body fluid to produce cations to promote blood clotting. The preferred oxyacid salts are alkali and alkaline earth salts of transition metals and halogen oxyacids with oxidizing capabilities sufficient to accelerate blood clotting. Another embodiment of the invention includes the compound containing an oxysalt plus a hydrophilic polymer such as carboxy methylcellulose, polyvinyl, alcohol, an alginate, and all soluble gums. Still another embodiment of the invention includes the compound formed of an oxyacid salt in combination with a hydrophilic proton donor and a solid desiccant which further accelerates blood coagulation reaction rates. The cation exchange material or an admixture of an alkali metal oxyacid salt plus acidic inorganic salt produces a scab or protective coating over the wound for protection and enhanced healing. Oxygen produced during the reaction substantially reduces the level of bacteria, virus and fungus at the wound.

It is therefore an object of this invention to provide a method of utilizing an oxyacid salt as a blood clotting agent for arresting blood flow from an open surface wound.

It is another object of this invention to provide a method of arresting blood and body fluid flow utilizing an oxyacid salt composition which is substantially sting-free when applied onto an open wound.

It is still another object of this invention to provide a composition utilizing an oxyacid salt Combined with an insoluble cation exchange material or an organic acidic or an inorganic salt to Arrest blood flow from an open skin wound.

Another object of this invention is to provide a composition of an oxyacid salt and an insoluble cation exchange material which, in addition to promoting blood clotting to arrest blood flow from an open wound, also provides antiseptic and a clumping or scabbing over the wound as a protective coating for the injury.

It is yet another object of this invention to provide a composition for promoting clotting of blood flowing from an open skin wound which minimizes or eliminates any sting associated with its application.

Still another object of this invention is to provide a localized rapid forming protective coating or covering that has antibacterial, antifungal and antiviral properties.

In accordance with these and other objects which will become apparent hereinafter, the instant invention will now be described.

### DETAILED DESCRIPTION OF THE INVENTION

Mechanism of Blood Coagulation

The following is offered as a brief explanation of one possible mechanism previously disclosed in U.S. Patent ______ which would explain the effectiveness of that related prior invention as described herebelow in full detail.

The plasma of circulating blood normally remains a liquid with its colloidal protein in the solid state. After albumin, the second most abundant protein in mammalian blood is a long, large molecule called fibrinogen. A series of enzymatic reactions take place during the clotting of blood. An inactive plasma enzyme (prothrombin) is converted into an active enzyme (thrombin) which, in turn, removes two pairs of amino acid groups from each fibrinogen molecule, converting into a molecule called fibrin monomer. Fibrin monomer then links together to form a polymer, which is the visible clot. The reactions can be summarized as follows:

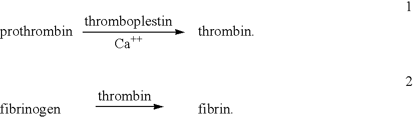

It is known that the decomposition of potassium ferrate produces the finest particles of iron oxide (Fe₂O₃) available. (See U.S. Pat. No. 4,545,974). Upon addition to water, K₂FeO₄ becomes Fe⁺⁺⁺ in the form of FeOOH, which upon drying, yields Fe₂O₃. The FeOOH (or Fe₂ O₃H₂O) is a solid in suspension and this ultra-fine material seems to be an ideal irritant for platelet membranes, thereby releasing the prothroplastin that is needed to initialize clotting. It is possible that they may tend to rupture the platelets themselves, thereby causing a massive release of clotting factors as does the rough surface of a wound achieve the same end.

It is possible that the Fe⁺⁺⁺ ion itself may aid in coagulation of blood. Trivalent ions, by lowering the zeta potential of a particle in solution, allow the particles (platelets) to clump more easily. Platelets are small disks of cytoplasm found in the blood of mammals. After a wound is received they begin to clump and stick around the wound area, causing the clumping and sticking of another cytoplasmic component, the thrombocycte. During this clumping process, certain phospholipids from the membrane of the platelets contribute to the overall clotting process, combined with the inactive plasma enzyme, Factor XII. Mechanical abrasion of the platelets is important in freeing the phospholipid component from the platelets.

Range of Useful Salt Ferrates

Initially, it was shown in U.S. Patent ______ that the utilization of potassium ferrate, again likely based upon the above-recited theory, effectively accomplishes the accelerated clotting of blood flowing from an open wound. The apparent chemical ferrate reaction with water found in blood was offered as follows:

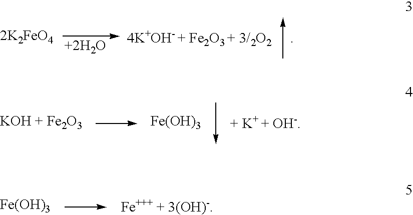

One of the important results therein was the production of the trivalent Fe⁺⁺⁺ ion which appeared to be the beneficial clotting agent provided in this aspect of that prior invention. Moreover, it was determined that the invention acted on all body fluids containing protein, such as that which flows from an open skin blister or burn.

A broadening of this aspect of that prior inventive compound was to substitute the potassium salt with others which possess the same cation properties as does the potassium cation. Those salt elements which will substitute for the potassium cation are shown in Tables I and II herebelow.

TABLE I

| | |
|---|---|
| H | Hydrogen |
| Li | Lithium |
| Na | Sodium |
| K | Potassium |
| Rb | Rubidium |
| Cs | Cesium |
| Fr | Francium |

TABLE II

| | | | | | |
|---|---|---|---|---|---|
| Be | Beryllium | Mg | Magnesium | Ca | Calcium |
| Sr | Strontium | Ba | Barium | Ra | Radium |
| Ti | Titanium | V | Vanadium | Cr | Chromium |
| Mn | Manganese | Fe | Iron | Co | Cobalt |
| Ni | Nickel | Cu | Copper | Zn | Zinc |
| Ga | Gallium | Ge | Geranium | Zr | Zirconium |
| Nb | Niobium | Mo | Molybdenum | Tc | Technetium |
| Ru | Ruthenium | Rh | Rhodium | Pd | Palladium |
| Ag | Silver | Cd | Cadmium | In | Indium |
| Sn | Tin | Hf | Hafnium | Ta | Tantalum |
| W | Tungsten | Re | Rhenium | Os | Osmium |
| Ir | Iridium | Pt | Platinum | Au | Gold |
| Hg | Mercury | Tl | Thallium | Pb | Lead |
| Bi | Bismuth | Al | Aluminum | As | Arsenic |
| NH₄ | Cation | N(C₄H₉)₄ | Cation | | |

In addition to the above salts in the cation form, all zeolites, sulfonated coal, and natural reoccurring membranes such as protein membranes will also act in compound form with ferrate to release the trivalent Fe⁺⁺⁺ ion to effect blood and body fluid coagulation.

Eliminating Stinging Effect

In utilizing the K₂FeO₄ as above described to arrest blood flow from a bleeding wound, equation 3 shows the presence of hydroxide (OH)⁻ radicals which are produced. The hydroxide (OH)⁻ radicals remain present in equation 5 and cause stinging at the wound site. Moreover, all of the cation salts of Table I produce the same result, i.e. stinging caused by the presence of the hydroxide ion.

All of the cation salts listed in Table II, however, produce a slightly altered chemical reaction which neutralizes all of the hydroxide ions produced. For example, using a calcium cation salt to replace the potassium cation causes the following chemical reaction with water in blood:

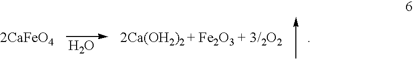

As can be observed from Equation 6, no hydroxide ions are produced. Rather, all are neutralized and combined with calcium as shown in the equation.

As provided by the above compounds, a method of arresting blood and body fluid flow from an open skin wound was provided. An effective amount of any of the above salt ferrates, and preferably potassium ferrate in powder form, was applied directly onto the wound to interact with flowing blood or body fluid to accelerate its clotting.

Salt Ferrate Combined with Resin

Although the above methodology and utilization of a salt ferrate greatly enhanced blood clotting, the wound nonetheless remained opened and generally unprotected unless the salt ferrate was combined with a carrier such as a BAND-AID, bandage, cotton member and the like which had been impregnated or coated with a dry powder taken from of one of the above chosen salt ferrate compositions.

By the addition of an ion exchange resin R with the salt ferrate, an additional benefit of scabbing or depositing of a substance produced by the reaction with water in the blood was accomplished over the open wound. Details of the composition and method of producing the preferred ion exchange resin R in the form of styrene divinylbenzene are disclosed in the previously referenced patents and are herein incorporated by reference. As described in formulas herebelow, the resin R may be shown in its chemical form or generally designated by the symbol “R” for simplicity. The ion exchange resin R was sulfonated as is shown in chemical terms in each chemical equation herebelow.

An acid form of the sulfonated ion exchange resin R is shown symbolically as follows:

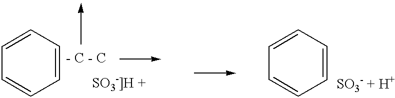

When the preferred hydrogen form of this sulfonated ion exchange resin R is in the presence of the salt ferrate and water within blood, the following reaction serves to neutralize the hydroxyl ions produced in equation 3 above.

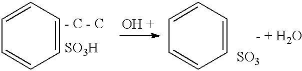

In addition to neutralizing hydroxyl ions by the presence of even trace amounts of the resin R to decrease or totally eliminate the stinging effect, excess trivalent Fe⁺⁺⁺ ions interact with the resin as follows:

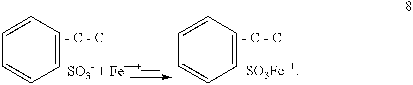

Thus, excess trivalent Fe⁺⁺⁺ charged ion cross links with the clotting blood in accordance with the following equation:

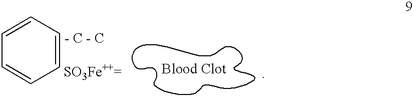

The amino acid in the blood protein are shown to interact with the resin:

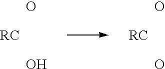

The K₂FeO₄ should be hygroscopic, small particles approximately 50 to 100 mesh size for best surface area. The ion exchange resin R is preferably in an acid form with some substitute Ca calcium ions as shown in equations 6 to 9. The cross linking of the resin R should be below 4.0 and as low as 0.25% and hygroscopic. The weight ratio should favor the dry ion exchange resin R by at least 4 to 1 of dry salt ferrate. The ion exchange resin R is preferably a cation exchange resin.

In another embodiment, a small amount of divalent calcium Ca++ may be added as an additional anticoagulant. Heparin-EDTA (Ethylene Dismine Tetracacitic Acid) potassium oxalate are anticoagulants and are ionic in action on the divalent Calcium Ca⁺⁺ and trivalent ion Fe⁺⁺⁺ to prevent clotting. By supplying excess of these ions, i.e. Fe⁺⁺⁺, clotting can be induced. Also, in addition to the hydrogen form of the resin R

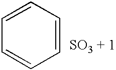

a given ratio of the calcium salt

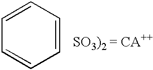

can supply excess of this ion to further induce blood clotting. The ferrate in contact with the blood-water on the skin forms O₂ which is a strong disinfectant to the cut.

### THE PRESENT INVENTION

Accelerated Blood Clotting

The present invention in one aspect thereof may be viewed as an expansion of the leaching of U.S. Patent ______ as outlined hereinabove. The present invention deals with the utilization of an inorganic acid containing oxygen known as an oxyacid in the salt form. Select oxyacid salts alone or in combinations as described herebelow, appear to have a similar beneficial effect upon accelerating the coagulation of blood and other protein based fluids flowing from an open wound.

The oxyacid salts which have been shown to produce this blood coagulation acceleration are as follows:

1. Alkali & alkaline earth salts;

2. Oxyacid salts of transition metals;

3. Halogen oxyacids;

4. Alkali & alkaline oxides, peroxides and superoxides.

Elimination of Sting

A hydrophilic proton donor may also be added which chemically combines to eliminate the sting caused by the presence of hydroxyl ions produced after the blood clotting reaction is in progress. In general, there are three categories of hydrophilic proton donors which will act as a matrix to accomplish the neutralization of the hydroxyl ions, where present, as follows:

1. Cation exchange resin (sulfonated, phosphorated or carbonated)

2. Acid producing salts

3. Organic acids.

Following are more specific examples of each of the three above-referenced general categories of compounds which will neutralize the hydroxyl acids present in the blood coagulation reaction of the present invention as follows:

1. Hydrogen form cation exchange resins (sulfonates)

2. Hydrogen form cation exchange resins (phosphonates)

3. Hydrogen form cation exchange resins (carbonates)

4. Acidic inorganic salts (e.g. NaHSO4)

5. Organic acids (e.g. Citric acid, carboxylic acids, amino acids, peptides, proteins)

6. Solid desiccants (e.g. CaCl₂, CaSO₄)

7. Porous hydrophilic matrix resins

8. Silicates (e.g. bentonite clay, hydroxy apatite)

9. Three component oxyacid, proton donor, solid desiccant

10. Polyvinyl alcohol

11. Carboxy methylcellulose

Solid desiccants also accelerate blood clotting further by water absorption from the blood.

Artificial Scab Formation

Another preferred function of the present invention is to create an artificial scab atop the open wound as the blood is more rapidly coagulated to arrest blood flow while also serving as an anti-microbial agent in the form of an oxidant. Such artificial scab forming agents fall into two general categories. The first category is that of a cation exchange material in combination with:

1. K₂FeO₄;

2. KMnO₄;

3. Na₂O₂;

4. KlO₃

5. K₂FeO₄+KMnO₄.

In addition to the above combinations with a cation exchange material, the compound formed as an admixture of

NaHSO₄+K₂FeO₄

as a unique combination of an oxyacid salt and an acidic inorganic salt, respectively, also provide this artificial scab-forming agent function.

The two major types of oxyacid salts, namely transition metal salts and halogen salt, act differently with respect to the scab-forming aspect of this invention. The transition metal oxyacid salts form metal oxides which are important in the matrix formation, or scab formation, when combined with the cation exchange material or any other hydrophilic proton donor. Halogen oxyacid salts do not possess this quality, nor do alkali or alkaline oxides, peroxides or superoxides. Although this later group does create an oxidizing environment that facilitates clotting, they do not act as efficiently as do the transition metal oxyacid salts to form a protective scab over the wound.

Blood Clot Testing Procedure

Preliminary blood clot testing was accomplished by forming a paraffin wax cake measuring approximately 30 mm in length, 3 mm in width, and 5 m deep trench in the paraffin. The trench was then filled with EDTA (Disodium Ethylenediaminetetraacetic salt) treated (0.1g/100 ml) bovine blood at ˜25C. Approximately 0.25 g of each coagulating agent in dry powder form was sprinkled over the trench as well as an additional area of about 5 mm beyond each edge of the trench in all directions. The approximate ratio of oxidant to the hydrogen form of a cation exchange resin as one test conducted was in a ratio of about 1:2, respectively.

After application of the coagulating agent atop the bovine blood, the treated blood remaining unclotted in the paraffin trench for about at least two hours, the strength of each scabbing matrix was qualitatively determined by placing a toothpick under the middle of the matrix and perpendicular to the length of the trench and then lifted for visual inspection. If the matrix remained intact, it was considered to have good matrix or scab-forming integrity. Each matrix was also inspected for tears, overall thickness and blood matrix integrity.

The following scab-forming agents were evaluated and found to have met this informal matrix integrity test:

1. Resin & K₂FeO₄

2. Resin & KMnO₄

3. Resin & Na₂O₂

4. Resin & KlO₃

5. Resin & K₂FeO₄ & KMnO₄

6. NaHSO₄ & K₂FeO₄.

While the instant invention has been shown and described herein in what are conceived to be the most practical and preferred embodiments, it is recognized that departures may be made therefrom within the scope of the invention, which is therefore not to be limited to the details disclosed herein, but is to be afforded the full scope of the claims so as to embrace any and all equivalent apparatus and articles.

What is claimed is:

1. A hemostatic agent adapted to be applied directly onto a bleeding wound comprising:
an effective amount of a oxyacid salt combined with an effective amount of an insoluble cation exchange material, said oxyacid salt combining with blood to promote blood clotting at the wound, said cation exchange material forming a protective cover over the wound as blood is thereby clotted.

2. A hemostatic agent as set forth in claim 1, wherein said oxyacid salt is taken from the group consisting of:
alkali and alkaline salts;
oxyacid salts of transition elements;
halogen oxyacids; and
alkali and alkaline oxides, peroxides and superoxides.

3. A hemostatic agent as set forth in claim 1, wherein said cation exchange material is an admixture which is a cation exchange resin and a compound taken from the group that includes:
K₂FeO₄;
KMₙO₄;
Na₂O₂; and
KlO₃FeO₄ and KMₙO₄.

4. A hemostatic agent as set forth in claim 1, wherein said cation exchange material includes:
K₂FeO₄ as said oxyacid salt;
NaHSO₄ as an acidic inorganic salt.

5. A method of arresting the flow of blood from a bleeding wound comprising the steps of:
A. providing an effective amount of a substantially anhydrous compound of an oxyacid salt combined with an effective amount of hydrophilic proton donor which will hydrate in the presence of blood to thereby promote clotting of the blood;
B. applying said compound to the wound for a time sufficient to effect sufficient clotting of the blood to arrest substantial further blood flow from the wound.

6. A method of arresting the flow of blood as set forth in claim 5, wherein said oxyacid salt is taken from the group consisting of:
alkali and alkaline salts;
oxyacid salts of transition elements;
halogen oxyacids; and
alkali and alkaline oxides, peroxides and superoxides.

7. A hemostatic agent adapted to be applied directly onto a bleeding wound comprising:
an effective amount of an oxyacid salt combined with an effective amount of a hydrophilic proton donor material, said oxyacid salt combining with blood to promote blood clotting at the wound, said hydrophilic proton donor material combining with, and thereby neutralizing, hydroxyl ions formed as said oxyacid salt combines with blood to effect clotting.

8. A hemostatic agent as set forth in claim 7, wherein said oxyacid salt is taken from the group consisting of:
alkali and alkaline salts;
oxyacid salts of transition elements;
halogen oxyacids; and
alkali and alkaline oxides, peroxides and superoxides.

9. A hemostatic agent as set forth in claim 7, wherein said hydrophilic proton donor is taken from the group that includes:
a cation exchange resin;
an acid producing salt; and
an organic acid.

10. A hemostatic agent as set forth in claim 7, further comprising:
a solid desiccant combined with said oxyacid salt and said hydrophilic proton donor material, said solid desiccant further accelerating blood clotting by absorbing water in the blood.

11. A hemostatic agent adapted to be applied directly onto a bleeding wound comprising:
an effective amount of an oxyacid salt combined with an effective amount of a hydrophilic polymer material, said oxyacid salt combining with blood to promote blood clotting at the wound, said hydrophilic polymer material forming a protective cover over the wound.

12. A hemostatic agent as set forth in claim 11, wherein said oxyacid salt is taken from the group consisting of:
alkali and alkaline salts;
oxyacid salts of transition elements;
halogen oxyacids; and
alkali and alkaline oxides, peroxides and superoxides.

13. A hemostatic agent as set forth in claim 12, wherein said hydrophilic polymer material is taken from the group that includes:
carboxy methylcellulose;
polyvinyl alcohol;
alginate;
gum aerobic; and
all soluble gums.
